(19) United States
(12) Patent Application Publication
(10) Pub. No.: US 2008/0299196 A1
(43) Pub. Date: Dec. 4, 2008

(54) Controlled Release Pharmaceutical Compositions Comprising a Fumaric Acid Ester

(75) Inventors: Henrik Nilsson, Copenhagen (DK); Bernd W. Muller, Flintbek (DE)

Correspondence Address: DARBY & DARBY P.C., P.O. BOX 770, Church Street Station, New York, NY, 10008-0770, US

(73) Assignee: Aditech Pharma AB, Malmo (SE)

(21) Appl. No.: 12/089,074

(22) PCT Filed: Oct. 6, 2006
(86) PCT No.: PCT/DK06/00561

§ 371 Date: Apr. 3, 2008

Related U.S. Application Data
(60) Provisional application No. 60/744,455, filed on Apr. 7, 2006.

(30) Foreign Application Priority Data
Oct. 7, 2005 (DK) PCT/DK2005/000648
Apr. 7, 2006 (DK) PA 2006 00510

(51) Int. Cl. A61K 9/22; A61K 31/225; A61P 17/06; A61P 3/10; A61P 1/00

(52) U.S. Cl. 424/468; 514/506

(57) ABSTRACT

The present invention relates to controlled release pharmaceutical compositions comprising fumaric acid ester(s) as active substance(s). The compositions are suitable for use in the treatment of e.g. psoriasis or other hyperproliferative, inflammatory or autoimmune disorders and are designed to release the fumaric acid ester in a controlled manner so that local high concentrations of the active substance within the gastrointestinal tract upon oral administration can be avoided and, thereby, enabling a reduction in gastro-intestinal related side-effects.

### FIELD OF THE INVENTION

The present invention relates to controlled release pharmaceutical compositions comprising a fumaric acid ester as an active substance. The compositions are suitable for use in the treatment of e.g. psoriasis or other hyperproliferative, inflammatory or autoimmune disorders and are designed to release the fumaric acid ester in a controlled manner so that local high concentrations of the active substance within the gastrointestinal tract upon oral administration can be avoided and, thereby, enabling a reduction in gastro-intestinal related side-effects.

### BACKGROUND OF THE INVENTION

Fumaric acid esters, i.e. dimethylfumarate in combination with ethylhydrogenfumarat have been used in the treatment of psoriasis for many years. The combination is marketed under the tradename Fumaderm®. It is in the form of tablets intended for oral use and it is available in two different dosage strengths (Fumaderm® initial and Fumaderm®):

| | Fumaderm ® Initial | Fumaderm ® |
|---|---|---|
| Dimethylfumarate | 30 mg | 120 mg |
| Ethylhydrogenfumarate, | 67 mg | 87 mg |
| calcium salt | | |
| Ethylhydrogenfumarate, | 5 mg | 5 mg |
| Magnesium salt | | |
| Etylhydrogenfumarate, | 3 mg | 3 mg |
| Zinc salt | | |

The two strengths are intended to be applied in an individually based dose regimen starting with Fumaderm® initial in an escalating dose, and then after e.g. three weeks of treatment switching to Fumaderm®. Both Fumaderm® initial and Fumaderm® are enteric coated tablets.

Another marketed composition is Fumaraat 120® containing 120 mg of dimethylfumarate and 95 mg of calcium monoethylfumarate (TioFarma, Oud-Beijerland, Netherlands). In a recent publication (Litjens et al. Br. J. Clin. Pharmacol. 2004, vol. 58:4, pp. 429-432), the pharmacokinetic profile of Fumaraat 120® is described in healthy subjects. The results show that a single oral dose of Fumaraat 120® is followed by a rise in serum monomethylfumarate concentration and only negligible concentrations of dimethylfumarate and fumaric acid is observed. The results indicate that dimethylfumarate is rapidly hydrolyzed to monomethylfumarate in an alkaline environment, but according to the authors not in an acid environment. As the composition is enteric coated, it is contemplated that the uptake of fumarate takes place mainly in the small intestine, where dimethylfumarate before uptake is hydrolysed to the monoester due to an alkaline environment, or it may rapidly be converted due to esterases in the circulation. Furthermore, the study shows that tₘₐₓ and Cₘₐₓ are subject to food effect, i.e. tₘₐₓ is prolonged (mean for fasted conditions is 182 min, whereas for fed conditions mean is 361 min) [lag time is 90 min for fasted and 300 min for fed] and Cₘₐₓ is decreased (fasted: 0.84 mg/l, fed: 0.48 mg/l) by concomitant food-intake. Another study (Reddingius W. G. Bioanalysis and Pharmacokinetics of Fumarates in Humans. Dissertation ETH Zurich No. 12199 (1997)) in healthy subjects with two tablets of Fumaderm® P forte revealed Cₘₐₓ values (determined as monoethyl- or monomethylfumarate) in a range from 1.0 to 2.4 μg/ml and a tₘₐₓ in a range of from 4.8 to 6.0 hours.

U.S. Pat. No. 6,277,882 and U.S. Pat. No. 6,355,676 disclose respectively the use of alkyl hydrogen fumarates and the use of certain fumaric acid mono alkyl ester salts for preparing micro tablets for treating psoriasis, psoriatic arthritis, neurodermatitis and enteritis regionalis Crohn. U.S. Pat. No. 6,509,376 discloses the use of certain dialkyl fumarates for the preparation of pharmaceutical preparations for use in transplantation medicine or the therapy of autoimmune diseases in the form of micro tablets or pellets. U.S. Pat. No. 5,424,332 discloses calcium, magnesium, zinc and iron salts of fumaric acid monoalkyl esters. U.S. Pat. No. 6,359,003 discloses treatment of transplant rejection by selectively suppressing host-versus-graft reaction using monoalkyl fumarate or its salt. U.S. Pat. No. 4,959,389 disclose compositions containing different salts of fumaric acid monoalkyl ester alone or in combination with dialkyl fumarate. GB 1,153,927 relates to medical compositions comprising dimethylmaleic anhydride and/or dimethylmaleic acid and/or a dimethylfumaric acid compounds. The Case report “Treatment of disseminated granuloma annulare with fumaric acid esters” from BMC Dermatology, vol. 2, no. 5, 2002, relates to treatment with fumaric acid esters. US 2004/0038889 discloses use of fumaric acid amides for the therapy of an autoimmune disease, mithchondrial diseases, and NF-kappaB mediated diseases and in transplantation medicine. WO 89/01830 discloses fumaric acid diamides and monoamides for treatment of psoriasis.

However, therapy with fumarates like e.g. Fumaderm® frequently gives rise to gastro-intestinal side effects such as e.g. fullness, diarrhea, upper abdominal cramps, flatulence and nausea.

Accordingly, there is a need to develop compositions comprising one or more therapeutically or prophylactically active fumaric acid esters that provide an improved treatment with a reduction in gastro-intestinal related side effects upon oral administration.

Furthermore, the present commercially available products contain a combination of two different esters of which one of the esters (namely the ethylhydrogenfumarate which is the monoethylester of fumaric acid) is present in three different salt forms (i.e. the calcium, magnesium and zinc salt). Although each individual form may have its own therapeutic profile it would be advantageous to have a much simpler product, if possible, in order to obtain a suitable therapeutic effect.

The present inventors contemplate that an improved treatment regimen may be obtained by administration of a pharmaceutical composition that is designed to deliver the active substance in a controlled manner, i.e. in a manner that is prolonged, slow and/or delayed compared with the commercially available product. Furthermore, it is contemplated that instead of using a combination of different fumaric acid esters, a suitable therapeutic response may be achieved by use of a single fumaric acid ester alone such as dimethylfumaric acid.

### SHORT DESCRIPTION OF THE FIGURES

FIG. 1 shows an example of an in vitro dissolution profile of a sample of a tablet (before the enteric coating is applied) prepared as described in example 1.

FIG. 2 shows an example of an in vitro dissolution profile of a sample of a tablet (before the enteric coating is applied) prepared as described in example 2.

FIG. 3 shows an example of an in vitro dissolution profile of a sample of a tablet (before the enteric coating is applied) prepared as described in example 3.

FIG. 4 shows an example of an in vitro dissolution profile of a sample of a tablet (before the enteric coating is applied) prepared as described in example 4.

FIG. 5 shows an example of an in vitro dissolution profile of a sample of a tablet (before the enteric coating is applied) prepared as described in example 5.

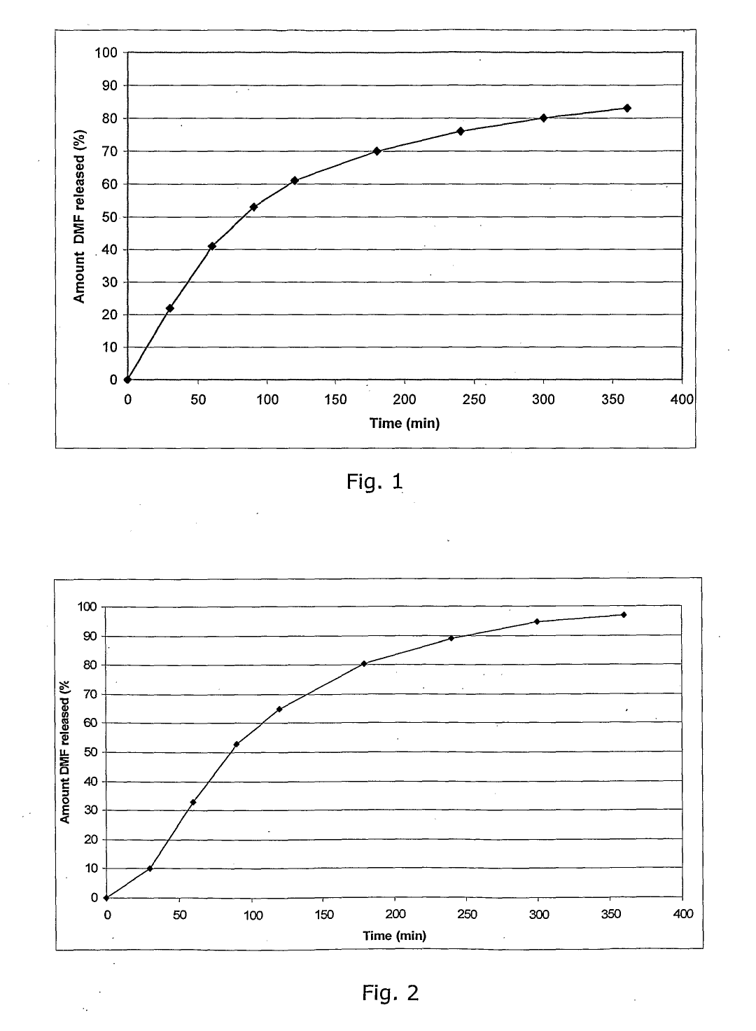

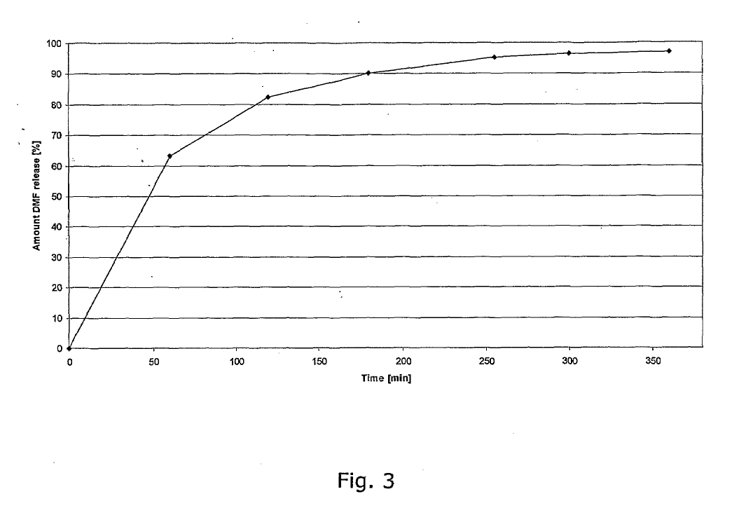

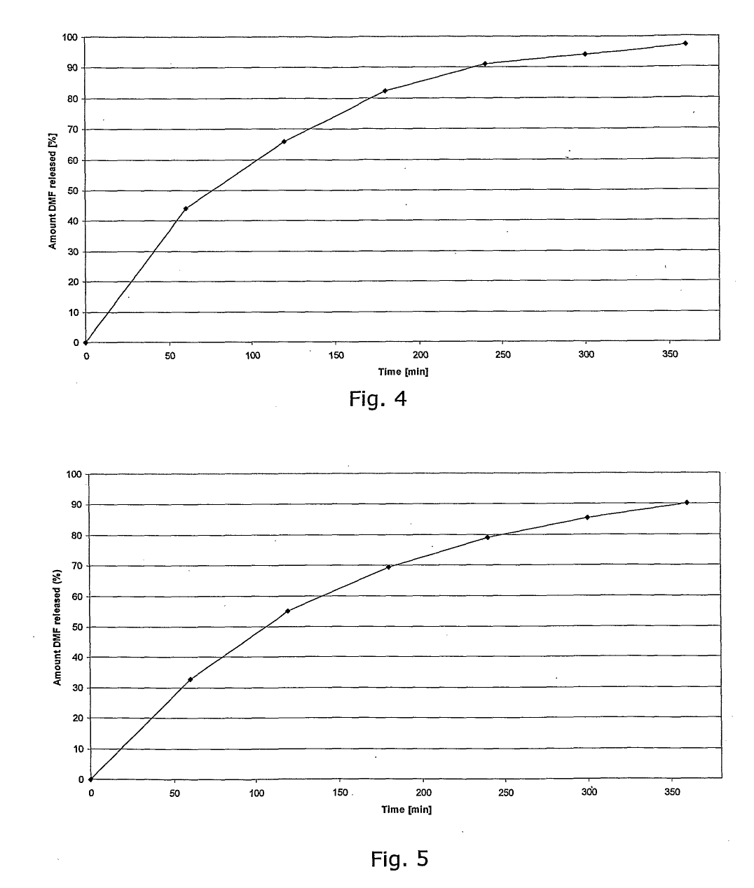

### SUMMARY OF THE INVENTION

The present invention provides in one aspect a pharmaceutical composition comprising as an active substance 40% to 50% by weight of one or more fumaric acid esters selected from di-(C₁-C₅)alkylesters of fumaric acid and mono-(C₁-C₅)alkylesters of fumaric acid, or a pharmaceutically acceptable salt thereof, 8% to 15% by weight of pharmaceutically acceptable polymer(s) and optionally pharmaceutically acceptable excipients or additives.

In a further aspect the invention provides a method of and the use of a pharmaceutical composition for the preparation of a medicament for treating psoriasis, psoriatic arthritis, neurodermatitis, inflammatory bowel disease, such as Crohn's disease and ulcerative colitis, polyarthritis, multiple sclerosis (MS), juvenile-onset diabetes mellitus, Hashimoto's thyroiditis, Grave's disease, SLE (systemic lupus erythematosus), Sjögren's syndrome, Pernicious anemia, Chronic active (lupoid) hepatitis, Rheumatoid arthritis (RA), lupus nephritis, myasthenia gravis, uveitis, refractory uveitis, vernal conjunctivitis, pemphigus vulgaris, scleroderma, optic neuritis, pain such as radicular pain, pain associated with radiculopathy, neuropathic pain or sciatica/sciatic pain, organ transplantation (prevention of rejection), sarcoidosis, necrobiosis lipoidica or granuloma annulare, which method comprises administering orally to a patient in need thereof, an effective dosage of a pharmaceutical composition according to the invention.

### DISCLOSURE OF THE INVENTION

As mentioned above, the present inventors contemplate that a suitable way of reducing the gastro-intestinal related side-effects is by administration of the active substance in the form of a controlled release composition.

It has now been found that a particular suitable controlled release composition is obtained when combining as an active substance one or more fumaric acid esters selected from di-(C₁-C₅)alkylesters of fumaric acid and mono-(C₁-C₅)alkylesters of fumaric acid, or a pharmaceutically acceptable salt thereof, a pharmaceutically acceptable polymer(s) and optionally pharmaceutically acceptable excipients or additives.

In one aspect, the invention relates to a pharmaceutical composition comprising as an active substance one or more fumaric acid esters selected from di-(C₁-C₅)alkylesters of fumaric acid and mono-(C₁-C₅)alkylesters of fumaric acid, or a pharmaceutically acceptable salt thereof, a pharmaceutically acceptable polymer(s) and optionally pharmaceutically acceptable excipients or additives.

In one aspect, the invention relates to a pharmaceutical composition comprising as an active substance 40% to 50% by weight of one or more fumaric acid esters selected from di-(C₁-C₅)alkylesters of fumaric acid and mono-(C₁-C₅)alkylesters of fumaric acid, or a pharmaceutically acceptable salt thereof, 8% to 15% by weight of pharmaceutically acceptable polymer(s) and optionally pharmaceutically acceptable excipients or additives.

In a further aspect of the invention, the amount of fumaric acid ester(s) is 42% to 48% by weight.

In another aspect of the invention, the amount of pharmaceutically acceptable polymer(s) is 9% to 14% by weight.

In a further aspect, the invention relates to a pharmaceutical composition further comprising from 0.1% to 5% by weight hydrophilic excipient(s).

In another aspect of the invention, the pharmaceutical composition comprises from 1% to 4% by weight hydrophilic excipient(s).

In a further aspect of the invention, the pharmaceutical composition is in the form of a tablet.

In a further aspect of the invention, the pharmaceutical composition is provided with an enteric coating.

In one aspect of the invention, the pharmaceutical composition is thus in the form of a tablet with an enteric coating having a pH controlled release (also known as a pH dependent release) of the fumaric acid ester. Normally, the release is designed so that only a small amount, if any, of the fumaric acid ester is released in the stomach (pH up to about 3), whereas the remainder, i.e. the majority of fumaric acid ester is released in the intestines (pH shifts to about 6-7). Such a pH controlled release can be obtained by providing a composition of the invention with an enteric coating (the whole composition or, if the composition is a multiparticulate composition, the individual units). Accordingly, the present invention relates in a further aspect to a controlled release pharmaceutical composition for oral use comprising as an active substance one or more fumaric acid esters selected from di-(C₁-C₅)alkylesters of fumaric acid and mono-(C₁-C₅)alkylesters of fumaric acid, or a pharmaceutically acceptable salt thereof, a pharmaceutically acceptable polymer(s) and optionally pharmaceutically acceptable excipients or additives, wherein the release of the fumaric acid ester—when subjected to an in vitro dissolution test employing 0.1 N hydrochloric acid as dissolution medium during the first 2 hours of the test and then 0.05 M phosphate buffer pH 6.5 as dissolution medium—is as follows:

within the first 3 hours after start of the test at the most about 50% w/w of the total amount of the fumaric acid ester contained in the composition is released, and/or
within the first 4 hours after start of the test at the most about 70% w/w of the total amount of the fumaric acid ester is released, and/or
within the first 5 hours after start of the test at the most about 85% w/w of the total amount of the fumaric acid ester is released, and/or
within the first 6 hours after start of the test at the most about 95% w/w of the total amount of the fumaric acid ester contained in the composition is released, and/or
within the first 7 hours after start of the test at the most about 98% w/w of the total amount of the fumaric acid ester contained in the composition is released, and/or
within the first 9 hours after start of the test at the most about 99% w/w of the total amount of the fumaric acid ester contained in the composition is released and/or
within the first 12 hours after start of the test at the most about 99% w/w of the total amount of the fumaric acid ester contained in the composition is released.

Accordingly, the present invention relates in yet a further aspect to a controlled release pharmaceutical composition for oral use comprising as an active substance 40% to 50% by weight of one or more fumaric acid esters selected from di-(C₁-C₅)alkylesters of fumaric acid and mono-(C₁-C₅)alkylesters of fumaric acid, or a pharmaceutically acceptable salt thereof, 8% to 15% by weight of pharmaceutically acceptable polymer(s) and optionally pharmaceutically acceptable excipients or additives, wherein the release of the fumaric acid ester—when subjected to an in vitro dissolution test employing 0.1 N hydrochloric acid as dissolution medium during the first 2 hours of the test and then 0.05 M phosphate buffer pH 6.5 as dissolution medium—is as follows:

within the first 3 hours after start of the test at the most about 50% w/w of the total amount of the fumaric acid ester contained in the composition is released, and/or
within the first 4 hours after start of the test at the most about 70% w/w of the total amount of the fumaric acid ester is released, and/or
within the first 5 hours after start of the test at the most about 85% w/w of the total amount of the fumaric acid ester is released, and/or
within the first 6 hours after start of the test at the most about 95% w/w of the total amount of the fumaric acid ester contained in the composition is released, and/or
within the first 7 hours after start of the test at the most about 98% w/w of the total amount of the fumaric acid ester contained in the composition is released, and/or
within the first 9 hours after start of the test at the most about 99% w/w of the total amount of the fumaric acid ester contained in the composition is released and/or
within the first 12 hours after start of the test at the most about 99% w/w of the total amount of the fumaric acid ester contained in the composition is released.

In the present context, a controlled release composition is a composition that is designed to release the fumaric acid ester in a prolonged, slow and/or delayed manner compared to the release of the commercially available product Fumaderm®, when tested under comparable conditions (e.g. for in vivo studies: dose equivalents, with or without standardized meal etc., or for in vitro studies: dose equivalents, dissolution test apparatus and working conditions including e.g. composition, volume and temperature of dissolution medium employed, rotation speed etc.).

The release in vivo may be tested by measuring the plasma concentration at predetermined time periods and thereby obtaining a plasma concentration versus time profile for the fumaric acid ester in question or, if relevant, a metabolite thereof. (E.g. in the case of dimethylfumarate, the active substance is envisaged to be methylhydrogenfumarate, i.e. the monomethyl ester of fumaric acid). Furthermore, it is contemplated that metabolism already takes place within the gastro-intestinal tract or during passage of the gastro-intestinal mucosa, or upon first passage through the hepatic circulation. Accordingly, when dimethylfumarate is administered, the relevant component to search for in the plasma may be the monomethyl ester and not the dimethylester of fumaric acid.

Other tests may also be used to determine or to give a measure of the release of the active substance in vivo. Thus, animals (e.g. mice, rats, dogs etc.) may be used as a model. The animals receive the compositions under investigation and after specified periods of time, the animals are sacrificed and the content of the active ingredient (or metabolite thereof, if relevant) is determined in plasma or specific organs or extracted from the intestinal contents. Another test involves the use of a specific segment of an animal intestine. The segment is placed in a suitable dissolution apparatus containing two compartments (a donor and a receiver) separated by the segment, and the composition under investigation is placed in a suitable medium in one compartment (the donor compartment). The composition will release the active substance that subsequently is transported across the intestinal segment. Accordingly, at suitable time intervals, the concentration of the active substance (or, if relevant, the metabolite) is measured in the receiver compartment.

A person skilled in the art will be able to adapt the above-mentioned method to the specific composition.

With respect to in vitro methods, well-established methods are available, especially methods described by official monographs like e.g. United States Pharmacopeia (USP) or the European Pharmacopoeia. A person skilled in the art will know which method to choose and how to select the specific conditions to carry out the in vitro test. For instance, the USP prescribes in vitro tests be carried out at 37+/−1.0 such as 37+/−0.5 degrees Celsius/Centigrade. A suitable dissolution test is, e.g as described in the United States Pharmacopoeia at 37° C. using a paddle dissolution apparatus at 100 rpm employing 0.1 N hydrochloric acid as dissolution medium during the first 2 hours of the test and then followed by 0.05 M phosphate buffer pH 6.5 as dissolution medium for the remaining test period.

As mentioned above, the in vivo release of the active substance is prolonged, slow and/or delayed compared with the commercially available Fumaderm® composition. In the present context, the term “prolonged” is intended to indicate that the active substance is released during a longer time period than Fumaderm® such as at least during a time period that is at least 1.2 times, such as, e.g., at least 1.5 times, at least 2 times, at least 3 times, at least 4 times or at least 5 times greater than that of Fumaderm®. Thus, if e.g. 100% of dimethylfumarate is released from Fumaderm® tablets 3 hours after the start of a suitable test, then 100% of dimethylfumarate in a composition according to the invention is released at least 3.6 hours after the start of a suitable test.

In the present context the term “delayed” is intended to indicate that the release of the active substance starts at a later point in time compared with that of Fumaderm® (such as at 30 min or more later such as, e.g., 45 min or more later, 1 hour or more later or 1.5 hours or more later, alternatively, that the initial release during the first 2 hours is much less compared with that of Fumaderm® (i.e. less than 80% w/w such as, e.g., less than 70% w/w, less than 60% w/w or less than 50% of that of Fumaderm®).

In one aspect, the composition according to the invention—upon oral administration and in comparison to that obtained after oral administration of Fumaderm® tablets in an equivalent dosage—gives a reduction in GI related side effects.

As used in the present invention, a gastrointestinal (GI) side effect may include, but is not limited to diarrhea, stomach ache, stomach pain, abdominal pain, abdominal cramps, nausea, flatulence, tenesmus, meteorism, an increased frequency of stools, a feeling of fullness and upper abdominal cramps.

In the present context, a reduction of GI related side effects is intended to denote a decrease in severity and/or incidence among a given treated patient population, compared to the GI side effects observed after administration of the composition according to the invention compared with that of Fumaderm®. A reduction in GI related side effects according to this definition could thus be construed as a substantial reduction in incidence of any of the GI side effect listed above, such as at least a 10% reduction in incidence or more preferably at least 20% reduction in incidence or even more preferable a more than 30% reduction in incidence. A reduction in GI related side effect can also be expressed as a substantial reduction in severity in any of the GI side effects listed above, such as a reduction in severity and/or frequency of diarrhea, stomach ache, stomach pain, abdominal pain, abdominal cramps, nausea, flatulence, tenesmus, meteorism, increased frequency of stools, a feeling of fullness or upper abdominal cramps. The reduction of GI related side effects, as described above, can be monitored in a clinical trial setting, either comparing the administration of the composition according to the invention head on with Fumaderm® or with placebo. In case of a placebo controlled trial, the incidence of GI related side effects in the patients receiving the composition according to the invention compared to the placebo group, can be compared to historical trials comparing Fumaderm® to placebo (see e.g. Altmeyer et al, J. Am. Acad. Dermatol. 1994; full reference: Altmeyer P J et al, Antipsoriatic effect of fumaric acid derivatives. Results of a multicenter double-blind study in 100 patients. J. Am. Acad. Dermatol. 1994; 30:977-81). Typically, patients suffering from psoriasis are included in such a study, and typically more than 10% of the body surface area will be affected by psoriasis (severe psoriasis). However, patients in whom between 2 and 10 percent of the body surface area is affected can also be included (moderate psoriasis). Patients can also be selected based on the psoriasis area severity index (PASI). Typically, patients within a certain range of PASI are included, such as between 10 and 40, or such as between 12 and 30, or such as between 15 and 25 or ≧10 or ≧12 or ≧16. Patients with any type of psoriasis may be included (chronic plaque type, exanthematic guttate type, pustular type, psoriatic erythroderma or palmoplantar type), but in some cases only patients with the chronic plaque type are included. About 15 to 20 patients in each treatment group (composition according to the invention and Fumaderm® or placebo) are sufficient in most cases, but more preferably about 30 to 50 patients are included in each arm of the study. Total study duration can be as short as one day to one week, but more preferably the study will run for 8 weeks to 12 weeks or up to 16 weeks. The side effects can e.g. be assessed as the total number of times a certain side effect was reported in each group (irrespective of how many patients have experienced the side effect), or the side effects can be assessed as the number of patients that have experienced a certain side effect a certain number of times, such as at least once or at least twice or at least three times during the duration of the study. Furthermore, the severity of a side effect can be monitored, or a certain severity of a side effect can be required for it to qualify as a side effect in the study. A convenient way of assessing the severity of a side effect is via a visual analogue (VAS) scale.

In a further aspect, the composition according to the invention—upon oral administration and in comparison to that obtained after oral administration of Fumaderm® tablets in an equivalent dosage—reduce flushing (frequency and/or severity).

In the present context the term “flushing” describes episodic attacks of redness of the skin together with a sensation of warmth or burning of the face, neck, and less frequently the upper trunk and abdomen. It is the transient nature of the attacks that distinguishes flushing from the persistent erythema of photosensitivity or acute contact reactions. Repeated flushing over a prolonged period of time can lead to telangiectasia and occasionally to classical rosacea of the face (Greaves M W. Flushing and flushing syndromes, rosacea and perioral dermatitis. In: Champion R H, et al, eds. Rook/Wilkinson/Ebling textbook of dermatology, 6^{th} ed., vol. 3. Oxford, UK: Blackwell Scientific, 1998: 2099-2104).

In the present context, a reduction of flushing is intended to denote a decrease in severity and/or incidence/frequency among a given treated patient population of flushing observed after administration of the composition according to the invention compared with flushing observed after administration of Fumaderm® and can be measured e.g as described by O'toole et al. Cancer 2000, 88(4): p. 770-776. A reduction in flushing according to this definition could thus be construed as a substantial reduction in incidence or severity of flushing. In one aspect of the invention, the incidence of flushing is reduced by at least about a third, in another aspect of the invention the incidence is reduced by half, and in a further aspect of the invention, the flushing incidence is reduced by about two thirds or more. Likewise, the severity is in one aspect of the invention reduced by at least about a third, in another aspect of the invention by at least half, and in a further aspect of the invention by at least about two thirds. Clearly a one hundred percent reduction in flushing incidence and severity is most preferable, but is not required. The reduction of flushing, as described above, can be monitored in a clinical trial setting, e.g. comparing the administration of the compound according to the invention compared with treatment with e.g. administration of Fumaderm®. In case of a Fumaderm® controlled trial, the incidence and severity, defined as mild, moderate or severe, of flushing in the patients receiving the compound according to the invention compared to the Fumaderm® group, can be compared. Typically, patients suffering from psoriasis are included in such a study, and typically more than 10% of the body surface area will be affected by psoriasis (severe psoriasis). However, patients in whom between 2 and 10 percent of the body surface area is affected can also be included (moderate psoriasis). Patients can also be selected based on the psoriasis area severity index (PASI). Typically, patients within a certain range of PASI are included, such as between 10 and 40, or such as between 12 and 30, or such as between 15 and 25 or ≧10 or ≧12 or ≧16. Patients with any type of psoriasis may be included (chronic plaque type, exanthematic guttate type, pustular type, psoriatic erythroderma or palmoplantar type), but in some cases only patients with the chronic plaque type are included. About 15 to 20 patients in each treatment group are sufficient in most cases, but more preferably about 30 to 50 patients are included in each arm of the study. Total study duration can be as short as one day to one week, but more preferably the study will run for 8 weeks to 12 weeks or up to 16 weeks. The side effects can e.g. be assessed as the total number of times a certain side effect was reported in each group (irrespective of how many patients have experienced the side effect), or the side effects can be assessed as the number of patients that have experienced a certain side effect a certain number of times, such as at least once or at least twice or at least three times during the duration of the study. Furthermore, the severity of a side effect can be monitored, or a certain severity of a side effect can be required for it to qualify as a side effect in the study. A convenient way of assessing the severity of a side effect is via a visual analogue (VAS) scale.

### Active Substance

The active substance in a composition of the invention is any fumaric acid ester. In one embodiment of the invention the fumaric acid ester is preferably selected from the group consisting of dimethylfumarate, diethylfumarate, dipropylfumarate, dibutylfumarate, dipentylfumarate, methyl-ethylfumarate, methyl-propylfumarate, methyl-butylfumarate, methyl-pentylfumarate, monomethylfumarate, monoethylfumarate, monopropylfumarate, monobutylfumarate and monopentylfumarate, including pharmaceutically acceptable salts thereof.

In a specific embodiment of the invention, the fumaric acid ester is a mono-(C₁-C₅)alkylester of fumaric acid that is present in the form of a pharmaceutically acceptable salt. Suitable salts are e.g. metal salts such as a salt selected from alkali metal salts and alkaline earth metal salts including sodium, potassium, calcium, magnesium or zinc salt.

The term (C₁-C₅)alkyl refers to a branched or un-branched alkyl group having from one to five carbon atoms inclusive, such as methyl, ethyl, 1-propyl, 2-propyl, 1-butyl, 2-butyl, 2-methyl-2-propyl, 2-methyl-1-propyl and pentyl.

In another embodiment, the composition according to the invention comprises dimethylfumarate as the active substance.

In a further embodiment, the composition according to the invention comprises monomethylfumarate as the active substance optionally in the form of a pharmaceutically acceptable salt like e.g. its sodium, potassium, calcium, magnesium and/or zinc salt.

In another embodiment, the composition according to the invention consists essentially of dimethylfumarate as the active substance.

In another embodiment, the composition according to the invention consists of dimethylfumarate as the active substance.

In a further embodiment, the composition according to the invention consists essentially of monomethylfumarate as the active substance optionally in the form of a pharmaceutically acceptable salt like e.g. its sodium, potassium, calcium, magnesium and/or zinc salt.

In a further embodiment, the composition according to the invention consists of monomethylfumarate as the active substance optionally in the form of a pharmaceutically acceptable salt like e.g. its sodium, potassium, calcium, magnesium and/or zinc salt.

In a further embodiment, the composition according to the invention comprises dimethylfumarate and monomethylfumarate (optionally in the form of a pharmaceutically acceptable salt like e.g. its sodium, potassium, calcium, magnesium and/or zinc salt) as the active substances, in a weight ratio between about 1:10 and about 10:1.

In a further embodiment, the composition according to the invention consists essentially of dimethylfumarate and monomethylfumarate (optionally in the form of a pharmaceutically acceptable salt like e.g. its sodium, potassium, calcium, magnesium and/or zinc salt) as the active substances, in a weight ratio between about 1:10 and about 10:1.

In a further embodiment, the composition according to the invention consists of dimethylfumarate and monomethylfumarate (optionally in the form of a pharmaceutically acceptable salt like e.g. its sodium, potassium, calcium, magnesium and/or zinc salt) as the active substances, in a weight ratio between about 1:10 and about 10:1.

Cosmetic and/or Pharmaceutical Compositions

The problem the invention solves is related to the appearance of gastro-intestinal side-effects upon oral administration of fumaric acid esters. By prolonging and/or delaying the release of the active substance from the composition it is envisaged that the local concentration of the active substance at specific sites of the gastro-intestinal tract is reduced (compared with that of Fumaderm®) which in turn leads to a reduction in gastro-intestinal side-effects. Accordingly, compositions that enable a prolonged and/or a slow release of a fumaric acid ester as defined above are within the scope of the present invention.

In specific embodiments, the invention relates to a controlled release pharmaceutical composition that may be administered one, two or more times daily, such as once or twice or three times daily. In one aspect of the invention, the composition is for administration once daily. In a further aspect of the invention, the composition is for administration twice daily. Furthermore, the composition may be designed so that it releases the fumaric acid ester relatively independent on pH, i.e. the release is not dependent on pH in the gastrointestinal tract. Examples of such compositions are e.g. compositions in the form of solid dosages forms (e.g. tablets, capsules, pellets, beads etc.) that are coated with a controlled release coating. Suitable materials for controlled release coatings are e.g. cellulose and cellulose derivatives including methylcellulose, ethylcellulose and cellulose acetate, or poly(ethylene-co-vinyl acetate), poly(vinyl chloride).

The release of the fumaric acid ester typically takes place in three steps from a composition coated with a diffusion controlled membrane:

i) firstly, water (from the GI tract) diffuses into the dosage form from the surroundings,
ii) secondly, at least some of the fumaric acid ester present in the dosage form dissolves by the action of water,
iii) the dissolved fumaric acid ester diffuses out of the dosage form and into the surroundings (i.e. the GI tract)

Other examples include e.g. matrix tablets or dosage form containing a multiplicity of units each in the form of a matrix system. The active substance is embedded in a matrix containing e.g. cellulose and cellulose derivatives including microcrystalline cellulose, hydroxypropyl methyl cellulose, hydroxypropyl cellulose and methylcellulose, povidone, poly(ethyleneoxide) (PEO), polyethylene glycol (PEG), poly(vinyl alcohol) (PVA), xanthan gum, carrageenan and other synthetic materials. Substances normally used as pharmaceutically acceptable excipients or additives may be added to a matrix composition. Other examples of suitable compositions are e.g. hydrogels, i.e. monolithic systems wherein the active substance is embedded in a water-swellable network polymer. Materials suitable for use include e.g. hydrophilic vinyl and acrylic polymers, polysaccharides like alginates, and poly(ethylene oxide).

As mentioned above, a composition according to the invention has in one aspect a pH controlled release (also known as a pH dependent release) of the fumaric acid ester. Normally, the release is designed so that only a small amount, if any, of the fumaric acid ester is released in the stomach (pH up to about 3), whereas the fumaric acid ester is released in the intestines (pH shifts to about 6-7). Such a pH controlled release can be obtained by providing a composition of the invention with an enteric coating (the whole composition or, if the composition is a multiparticulate composition, the individual units).

Examples of suitable substances for use as enteric coating materials include polyacrylamides, phthalate derivatives such as acid phthalates of carbohydrates, amylose acetate phthalate, cellulose acetate phthalate, other cellulose ester phthalates, cellulose ether phthalates, hydroxypropylcellulose phthalate, hydroxypropylethylcellulose phthalate, hydroxypropylmethylcellulose phthalate, methylcellulose phthalate, polyvinyl acetate phthalate, poly acrylic methacrylic acid copolymers, shellac and vinyl acetate and crotonic acid copolymers, etc.

Furthermore, the compositions may be formulated in such a manner that an initial delay in release of the fumaric acid ester is obtained. Such a delay may be obtained e.g. by choosing an outermost coating that in a time-controlled manner degrades (e.g. erodes) and only when this outermost coating is eroded away, the release of the fumaric acid ester starts.

In the following is given a description of specific embodiments, wherein the fumaric acid ester is released depending on pH and wherein the release pattern is suitable for compositions that are administered two or more times daily. Examples of suitable formulation principles are e.g. compositions provided with an enteric coating or hydrogels of a type described by Zentner et al (U.S. Pat. No. 6,537,584) and Bae (U.S. Pat. No. 5,484,610), which hereby are incorporated by reference. Further examples of suitable formulation principles are e.g. compositions provided with a diffusion coating such as a controlled release diffusion coating, matrix particulates or matrix tablets, hydrogels, pulsed dose drug delivery systems, co-formulation with vitamin E concentrate or ethanol, TPGS, corn oil and wax etc., including any of the formulation principles mentioned above, optionally with an enteric coating.

In another further aspect of the invention, a controlled release pharmaceutical composition comprising as an active substance one or more fumaric acid esters selected from di-(C₁-C₅)alkylesters of fumaric acid and mono-(c₁-C₅)alkylesters of fumaric acid, or a pharmaceutically acceptable salt thereof, a pharmaceutically acceptable polymer(s) and optionally pharmaceutically acceptable excipients or additives, characterized in that it consists of a controlled-release dosage form adapted to release di-(C₁-C₅)alkylester and/or a mono-(C₁-C₅)alkylester of fumaric acid or a pharmaceutically acceptable salt thereof over a predetermined time period, according to an in vitro profile of dissolution when measured according to USP in 0.1 N hydrochloric acid during the first 2 hours and then 0.05 M phosphate buffer at a pH 6.5 or 6.8,

wherein at the most 5% w/w of the total amount of the fumaric acid ester contained in the composition is released within the first 2 hours after start of the test and/or,
wherein from about 20% to about 50% w/w of the total amount of the fumaric acid ester contained in the composition is released within the first 3 hours after start of the test, and/or
wherein from about 45% to about 70% w/w of the total amount of the fumaric acid ester contained in the composition is released within the first 4 hours after start of the test, and/or
wherein from about 65% to about 85% w/w of the total amount of the fumaric acid ester contained in the composition is released within the first 5 hours after start of the test, and/or
wherein from about 75% to about 90% w/w of the total amount of the fumaric acid ester contained in the composition is released within the first 6 hours after start of the test, is provided.

In yet another aspect of the invention, a controlled release pharmaceutical composition comprising as an active substance one or more fumaric acid esters selected from di-(C₁-C₅)alkylesters of fumaric acid and mono-(C₁-C₅)alkylesters of fumaric acid, or a pharmaceutically acceptable salt thereof, a pharmaceutically acceptable polymer(s) and optionally pharmaceutically acceptable excipients or additives, characterized in that it consists of a controlled-release dosage form adapted to release di-(C₁-C₅)alkylester and/or a mono-(C₁-C₅)alkylester of fumaric acid or a pharmaceutically acceptable salt thereof over a predetermined time period, according to a in vitro profile of dissolution when measured according to USP in 0.1 N hydrochloric acid during the first 2 hours and then 0.05 M phosphate buffer at a pH 6.5 or 6.8, wherein at the most 5% w/w of the total amount of the fumaric acid ester contained in the composition is released within the first 2 hours after start of the test, wherein from about 20% to about 50% w/w of the total amount of the fumaric acid ester contained in the composition is released within the first 3 hours after start of the test, wherein from about 45% to about 70% w/w of the total amount of the fumaric acid ester contained in the composition is released within the first 4 hours after start of the test, wherein from about 65% to about 85% w/w of the total amount of the fumaric acid ester contained in the composition is released within the first 5 hours after start of the test, wherein from about 75% to about 90% w/w of the total amount of the fumaric acid ester contained in the composition is released within the first 6 hours after start of the test, and wherein at least 80% of the total amount of the fumaric acid ester contained in the composition is released within the first 7 hours after start of the test, is provided.

In yet another aspect of the invention, a controlled release pharmaceutical composition comprising as an active substance 40% to 50% by weight of one or more fumaric acid esters selected from di-(C₁-C₅)alkylesters of fumaric acid and mono-(C₁-C₅)alkylesters of fumaric acid, or a pharmaceutically acceptable salt thereof, 8% to 15% by weight of pharmaceutically acceptable polymer(s) and optionally pharmaceutically acceptable excipients or additives, characterized in that it consists of a controlled-release dosage form adapted to release di-(C₁-C₅)alkylester and/or a mono-(C₁-C₅)alkylester of fumaric acid or a pharmaceutically acceptable salt thereof over a predetermined time period, according to a in vitro profile of dissolution when measured according to USP in 0.1 N hydrochloric acid during the first 2 hours and then 0.05 M phosphate buffer at a pH 6.5 or 6.8, wherein at the most 5% w/w of the total amount of the fumaric acid ester contained in the composition is released within the first 2 hours after start of the test, wherein from about 20% to about 50% w/w of the total amount of the fumaric acid ester contained in the composition is released within the first 3 hours after start of the test, wherein from about 45% to about 70% w/w of the total amount of the fumaric acid ester contained in the composition is released within the first 4 hours after start of the test, wherein from about 65% to about 85% w/w of the total amount of the fumaric acid ester contained in the composition is released within the first 5 hours after start of the test, wherein from about 75% to about 90% w/w of the total amount of the fumaric acid ester contained in the composition is released within the first 6 hours after start of the test, is provided.

In yet another aspect of the invention, a controlled release pharmaceutical composition comprising as an active substance one or more fumaric acid esters selected from di-(C₁-C₅)alkylesters of fumaric acid and mono-(C₁-C₅)alkylesters of fumaric acid, or a pharmaceutically acceptable salt thereof, a pharmaceutically acceptable polymer(s) and optionally pharmaceutically acceptable excipients or additives, characterized in that it consists of a controlled-release dosage form adapted to release di-(C₁-C₅)alkylester and/or a mono-(C₁-C₅)alkylester of fumaric acid or a pharmaceutically acceptable salt thereof over a predetermined time period, according to a in vitro profile of dissolution when measured according to USP in 0.1 N hydrochloric acid during the first 2 hours and then 0.05 M phosphate buffer at a pH 6.5 or 6.8, wherein at the most 5% w/w of the total amount of the fumaric acid ester contained in the composition is released within the first 2 hours after start of the test, wherein from about 20% to about 50% w/w of the total amount of the fumaric acid ester contained in the composition is released within the first 3 hours after start of the test, wherein from about 45% to about 70% w/w of the total amount of the fumaric acid ester contained in the composition is released within the first 4 hours after start of the test, wherein from about 65% to about 85% w/w of the total amount of the fumaric acid ester contained in the composition is released within the first 5 hours after start of the test, wherein from about 75% to about 95% w/w of the total amount of the fumaric acid ester contained in the composition is released within the first 6 hours after start of the test, and wherein at least 80% of the total amount of the fumaric acid ester contained in the composition is released within the first 7 hours after start of the test, is provided.

In yet another aspect of the invention, a controlled release pharmaceutical composition comprising as an active substance 40% to 50% by weight of one or more fumaric acid esters selected from di-(C₁-C₅)alkylesters of fumaric acid and mono-(C₁-C₅)alkylesters of fumaric acid, or a pharmaceutically acceptable salt thereof, 8% to 15% by weight of pharmaceutically acceptable polymer(s) and optionally pharmaceutically acceptable excipients or additives, characterized in that it consists of a controlled-release dosage form adapted to release di-(C₁-C₅)alkylester and/or a mono-(C₁-C₅)alkylester of fumaric acid or a pharmaceutically acceptable salt thereof over a predetermined time period, according to a in vitro profile of dissolution when measured according to USP in 0.1 N hydrochloric acid during the first 2 hours and then 0.05 M phosphate buffer at a pH 6.5 or 6.8, wherein at the most 5% w/w of the total amount of the fumaric acid ester contained in the composition is released within the first 2 hours after start of the test, wherein from about 20% to about 50% w/w of the total amount of the fumaric acid ester contained in the composition is released within the first 3 hours after start of the test, wherein from about 45% to about 70% w/w of the total amount of the fumaric acid ester contained in the composition is released within the first 4 hours after start of the test, wherein from about 65% to about 85% w/w of the total amount of the fumaric acid ester contained in the composition is released within the first 5 hours after start of the test, wherein from about 75% to about 95% w/w of the total amount of the fumaric acid ester contained in the composition is released within the first 6 hours after start of the test, is provided.

Typically, as described above, the compositions according to the invention are designed to deliver the active substance (i.e. the monoalkylester of fumaric acid, which in turn is metabolised to fumaric acid and, which subsequently is subjected to a rapid elimination process) in a prolonged manner. Apart from the characteristic in vitro release patterns described herein, such a prolonged release is reflected in the pharmacokinetic parameters obtained after a clinical study as well. Accordingly, it is contemplated that the Cₘₐₓ of the monoalkylester of fumaric acid (which appears in the plasma upon hydrolysis or metabolism of the dialkylester administered) is of the same order of magnitude as previously described in the literature provided that a similar or equivalent dose is administered (i.e. Cₘₐₓ of monomethylfumarate in a range of from about 0.4 to about 2.0 mg/l corresponding to an oral dose of 120 to 240 mg dimethylfumarate). However, in order to avoid many frequent daily administrations (2-4 tablets 1-3 times daily) it is an aim to prolong the time period where the concentration is within the therapeutic window. Accordingly, it is contemplated that W₅₀ (i.e. the time period in which the plasma concentration is 50% of Cₘₐₓ or more) is prolonged compared to the marketed treatment with at least 10% such as, e.g. at least 20%, at least 30%, at least 40% or at least 50%. A suitable W₅₀ is believed to be at least 2 hours such as in a range of from about 2 to about 15 hours or from about 2.5 to about 10 hours or from about 3 to about 8 hours.

Furthermore, it is contemplated that a controlled release composition according to the invention may lead to a reduced interindividual and/or intraindividual variation in the plasma profile and to a reduced dependency on whether the composition is taken together with or without food (a reduced variation of the plasma concentration profile of monomethylfumarate when the pharmaceutical composition is administered with or without concomitant food intake). Therefore, the controlled release composition according to the invention may lead to a reduced frequency of dosing and/or a reduced average total daily dose, and/or an increased efficacy at the same total daily dose of the active substance compared to Fumaderm®.

Different kinetic models, such as zero-order (1), first-order (2), square-root (Higuchi's equation) (3) can be applied to the interpretation of the drug release kinetic.

Mₜ=M₀+k₀*t 1

ln Mₜ=ln M+k₁*t 2

Mₜ=M₀+k_{H}*t^{1/2} 3

In these equations, Mₜ is the cumulative amount of drug released at any specified time point and M₀ is the dose of active substance incorporated in the pharmaceutical composition. k₀, k₁ and k_{H} are rate constants for zero-order, first-order and Higuchi's equation, respectively.

One aspect of the invention relates to a zero-order dissolution release profile. Another aspect relates to a first-order dissolution release profile. A further aspect relates to a square-root (Higuchi's equation) dissolution release profile.

In one aspect of the invention, the pharmaceutically acceptable polymer is a film-binding polymer. Examples of “pharmaceutically acceptable polymer(s)” comprises but are not limited to one or more selected from the group consisting of ethylcellulose (e.g. Ethocel® NF), methacrylic acid copolymer, and acrylic acid copolymer, such as ammonio methacrylate copolymer type A (e.g. Eudragit® RL30D) and/or B or methacrylic acid copolymer A and/or B. Other examples comprise but are not limited to polyvinyl acetate polymer (e.g. Kollicoat SR30D) and methacryl acetate polymer (e.g. Kollicoat MAE 30DP).

In one aspect of the invention, examples of “pharmaceutically acceptable polymer(s)” comprise but are not limited to one or more selected from the group consisting of ethylcellulose (e.g. Ethocel® NF), methacrylic acid copolymer such as methacrylic acid copolymer A and/or B, acrylic acid copolymer such as vinyl-pyrrolidone acrylic acid copolymer and ethyl acrylic acid copolymer, and ammonio methacrylate copolymer, such as ammonio methacrylate copolymer type A (e.g. Eudragit® RL30D) and/or B (e.g. Eudragit® RS30D). Other examples comprise but are not limited to polyvinyl acetate polymer (e.g. Kollicoat SR30D) and methacryl acetate polymer (e.g. Kollicoat MAE 30DP).

In one aspect of the invention, the pharmaceutically acceptable polymer is one or more selected from the group consisting of ethylcellulose, methacrylic acid copolymer, and acrylic acid copolymer.

In a further aspect of the invention, the pharmaceutically acceptable polymer is one or more selected from the group consisting of ammonio methacrylate copolymer type A, ammonio methacrylate copolymer B, methacrylic acid copolymer A, methacrylic acid copolymer B, polyvinyl acetate polymer and methacryl acetate polymer.

In a further aspect of the invention, the pharmaceutically acceptable polymer is ammonio methacrylate copolymer type A.

In a further aspect of the invention, the pharmaceutically acceptable polymer is ammonio methacrylate copolymer B.

In a further aspect of the invention, the pharmaceutically acceptable polymer is methacrylic acid copolymer A.

In a further aspect of the invention, the pharmaceutically acceptable polymer is methacrylic acid copolymer B.

In a further aspect of the invention, the pharmaceutically acceptable polymer is polyvinyl acetate polymer.

In a further aspect of the invention, the pharmaceutically acceptable polymer is methacryl acetate polymer.

Examples of “hydrophilic excipient(s)” comprises but are not limited to polyethylene glycol (PEG), povidone, hydroxyl propyl cellulose (HPC), hydroxyethyl starch (HES) or hydroxypropyl methyl cellulose (HPMC) or a material with similar properties, or a combination thereof.

In a further aspect of the invention, a controlled release pharmaceutical composition, wherein the pharmaceutically acceptable polymer is ethyl cellulose, is provided. In a further aspect of the invention, a controlled release pharmaceutical composition, wherein the pharmaceutically acceptable polymer is methacrylic acid copolymer A, is provided.

In another aspect of the invention a controlled release pharmaceutical composition, wherein the hydrophilic excipient is hydroxyl propyl cellulose, is provided.

In another aspect of the invention, a controlled release pharmaceutical composition, wherein the hydrophilic excipient is polyethylene glycol, is provided.

Tablets are typically prepared by direct compression, by wet granulation, or by dry granulation. Their formulations usually incorporate pharmaceutically acceptable excipients or additives such as diluents, binders, lubricants, surfactants, glidants, disintegrators (dissolving accelerator), and plasticiser (softener). Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like.

Polyethylene glycol, ethylcellulose and waxes can also serve as binders.

A lubricant may be necessary in a tablet formulation to prevent the tablet and punches from sticking in the die. The lubricant may be chosen from such slippery solids as talc, magnesium and calcium stearate, stearic acid and hydrogenated vegetable oils. Tablet disintegrators are substances which swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethylcellulose, for example, may be used, as well as sodium lauryl sulfate.

Triethylcitrate, acetyl-triethylcitrate, tributylcitrate, acetyl-tributylcitrate, tricetin, diethylphthalate, dibiutysebacate, dibutylphthlate, polyethylengycole, glycerole, and polysorbate may be used as softeners. One of the functions of the softener is to decrease the glass transition point of the polymer and this will lead to lower film formation temperatures.

Exemplary surfactants and surface active agents may be selected from known pharmaceutical excipients such as, for example, gelatin, casein, lecithin, gum acacia, stearic acid or other fatty acids, benzalkonium chloride, calcium stearate, glyceryl monostearate or other fatty acid salts, polyethylene glycols, silicon dioxide, methylcelluloses or carboxymethylcelluloses, sodium stearyl fumarate, magnesium stearate, alginate, or any other surface modifying compounds known in the art.

Enteric formulations are often used to protect an active ingredient from the strongly acid contents of the stomach. Such formulations can be created by coating a solid dosage form with a film of a polymer which is insoluble in acid environments, and soluble in basic environments. Exemplary films are cellulose acetate phthalate, polyvinyl acetate phthalate, hydroxypropyl methylcellulose phthalate and hydroxypropyl methylcellulose acetate succinate.

In order to prevent the edges of a tablet from breaking, the surface of the tablets may be cured. For that purpose the tablets can be coated with a solution of 5% Kollidon 25 in an amount of 1 to 2 mg/cm² tablet surface. The PVP (polyvinylpyrrolidon)-Solution is, depending on the process (fluid bed, drageecauldron), water-, water/isopropanol- or isopropanol-based.

Various controlled release formulations, not limiting the scope of the present invention, illustrating the invention are described hereafter (all concentrations based on the final tablet):

### Granules

In one aspect, granules may be prepared by mixing and/or granulating the active substance at a concentration of about 10 to about 90%, such as of about 20 to about 80%, of about 30 to about 75%, or of about 40 to about 70%, especially from about 50 to about 70%, with granulating excipients, such as pharmaceutical acceptable polymers, e.g. ethylcellulose such as Ethocel® NF premium, or methacrylic/acrylic acid copolymers or ammonio methacrylate copolymers, such as ammonio methacrylate copolymer type A or B, or methacrylic acid copolymer A or B, or polyvinyl acetate polymer, or methacryl-ethylacetate polymer, incorporated at a concentration between about 8 to about 15%. Hydrophilic excipients such as polyethylene glycol (PEG), povidone, hydroxyl propyl cellulose (HPC), hydroxyethyl starch (HES) or hydroxypropyl methyl cellulose (HPMC) at a concentration of about 0.1 to about 5% and/or pharmaceutical acceptable surfactants with HLB values above 8 at a concentration of about 0.01 to about 3% may be incorporated.

### Tablets

Tablets may be based on granules. When it comes to producing tablets in large scale, especially on a rotary machine, further excipients to increase flow ability or to improve tabletting-behaviour may be needed. As filling and binding excipients, if required, mention can be made of e.g. microcrystalline cellulose, such as Avicel® 102, and cellulose at a concentration of about 1 to about 60%, crystalline, spray dried or granulated lactose monohydrate e.g. Tablettose®, as well as anhydrous lactose, at a concentration of about 5 to about 60%, sugar alcohols, such as sorbitol and mannitol, at a concentration of about 0 to about 40% and modified starch at a concentration of about 0 to about 40%. Furthermore disintegration agents such as starch and starch-derivates such as sodium starch glycolate (at a concentration of about 0.2 to about 10%), crospovidone (at a concentration of about 0.2 to about 10%), sodium carboxymethylcellulose (at a concentration of about 0.1 to about 10%), glidants such as colloidal anhydrous and hydrous silica (at a concentration of about 0.2 to about 4%), and lubricants, e.g. magnesium stearate, calcium behenate, and calciumarachinate (at a concentration of about 0.2 to about 3%) or sodium stearyl fumarate (at a concentration of about 1 to about 8%) can be added.

In one aspect, tablets may be based on a mixture of granules comprising active substance and placebo granules without active substance.

### Dosage

Apart from providing compositions having different content of fumaric acid present, the invention also provides e.g. kits containing two or more containers e.g. with compositions having various amounts of the fumaric acid included. Such kits are suitable for use in those situations where an increasing dosage is required over time. A normal up-scale of the dosage is given below:

| Week | Morning | Noon | Evening | Strength |
|---|---|---|---|---|
| 1 | 1 | — | — | A |
| 2 | 1 | — | 1 | A |
| 3 | 1 | — | 1 | B |
| 4 | 1 | — | — | B |
| 5 | 1 | — | 1 | B |
| 6 | 1 | 1 | 1 | B |
| 7 | 2 | 1 | 1 | B |
| 8 | 2 | 1 | 2 | B |
| 9 | 2 | 2 | 2 | B |

A corresponds to a low strength such as about 30 mg dimethylfumarate (or a corresponding effective dose of another fumaric acid ester)
B corresponds to a higher strength such as about 120 mg dimethylfumarate (or a corresponding effective dose of another fumaric acid ester)

In one aspect of the invention, a controlled release pharmaceutical composition is provided wherein the amount of one or more fumaric acid esters selected from di-(C₁-C₅)alkylesters of fumaric acid and mono-(C₁-C₅)alkylesters of fumaric acid, or a pharmaceutically acceptable salt thereof, in a dosage form is from 50 mg to 1000 mg active substance, such as 90 mg to 600 mg active substance.

In one aspect of the invention, a controlled release pharmaceutical composition, wherein the amount of one or more fumaric acid esters selected from di-(C₁-C₅)alkylesters of fumaric acid and mono-(C₁-C₅)alkylesters of fumaric acid, or a pharmaceutically acceptable salt thereof, in a dosage form is from 90 mg to 500 mg active substance, such as 90 mg to 360 mg active substance, such as 90, 120, 180, 240, 360, 450, 480, or 500 mg active substance, is provided. In a further aspect of the invention the amount of active substance is 120, 180 or 240 mg active substance. In yet a further aspect of the invention, the amount of active substance is 180 or 360 mg.

In yet a further aspect of the invention, the amount of active substance in a dosage form is 120 mg active substance. In yet a further aspect of the invention, the amount of active substance in a dosage form is 180 mg active substance. In yet a further aspect of the invention, the amount of active substance in a dosage form is 240 mg active substance. In yet a further aspect of the invention, the amount of active substance in a dosage form is 360 mg active substance. In yet a further aspect of the invention, the amount of active substance in a dosage form is 450 mg active substance. In yet a further aspect of the invention, the amount of active substance in a dosage form is 480 mg active substance. In yet a further aspect of the invention, the amount of active substance in a dosage form is 500 mg active substance.

In yet a further aspect of the invention, the amount of active substance in a dosage form is 50 mg active substance. In yet a further aspect of the invention, the amount of active substance in a dosage form is 100 mg active substance. In yet a further aspect of the invention, the amount of active substance in a dosage form is 150 mg active substance. In yet a further aspect of the invention, the amount of active substance in a dosage form is 200 mg active substance. In yet a further aspect of the invention, the amount of active substance in a dosage form is 250 mg active substance. In yet a further aspect of the invention, the amount of active substance in a dosage form is 300 mg active substance. In yet a further aspect of the invention, the amount of active substance in a dosage form is 350 mg active substance. In yet a further aspect of the invention, the amount of active substance in a dosage form is 400 mg active substance. In yet a further aspect of the invention, the amount of active substance in a dosage form is 450 mg active substance. In yet a further aspect of the invention, the amount of active substance in a dosage form is 600 mg active substance. In yet a further aspect of the invention, the amount of active substance in a dosage form is 700 mg active substance. In yet a further aspect of the invention, the amount of active substance in a dosage form is 800 mg active substance. In yet a further aspect of the invention, the amount of active substance in a dosage form is 900 mg active substance. In yet a further aspect of the invention, the amount of active substance in a dosage form is 1000 mg active substance.

The daily dosage of the controlled release pharmaceutical composition according to the invention that is administered to treat a patient depends on a number of factors among which are included, without limitation, weight and age and the underlying causes of the condition or disease to be treated, and is within the skill of a physician to determine. In one aspect of the invention the daily dosage can be e.g. from 240 to 360 mg active substance given in one to three doses, in another aspect from 360 to 480 mg active substance given in one to three doses, in another aspect 480 to 600 mg active substance given in one to three doses, in another aspect 600 to 720 mg active substance given in one to three doses, in another aspect 720 to 840 mg active substance given in one to three doses, in another aspect 840 to 960 mg active substance given in one to three doses, in yet another aspect 960 to 1080 mg active substance given in one to three doses, and in yet another aspect 700 to 1500 mg active substance given in one to three doses.

In another aspect of the invention the controlled release pharmaceutical composition in the form of a tablet is provided, such as a tablet which has a shape that makes it easy and convenient for a patient to swallow e.g. a tablet which has a rounded or a rod-like shape without any sharp edges.

In another aspect of the invention a pharmaceutical composition in the form of a tablet designed to be divided into two or more parts, is provided.

The compositions according to the invention may be administered together with a meal or in relation to a meal such as e.g. in a time period corresponding to a range from at least about 30 minutes before a meal to about 2 hours after the meal, or the composition may be administered at any specific point(s) in time during the day.

In one embodiment, the total daily dose is given at bedtime, such as up to or about 30 minutes before bedtime, up to or about 60 minutes before bedtime, up to or about 90 minutes before bedtime, up to or about 120 minutes before bedtime or up to or about 180 minutes before bedtime.

The compositions and kits according to the invention are contemplated to be suitable to use in the treatment of one or more of the following conditions:
a. Psoriasis
b. Psoriatic arthritis
c. Neurodermatitis
d. Inflammatory bowel disease, such as
   i. Crohn's disease
   ii. Ulcerative colitis
e. Polyarthritis
f. Multiple sclerosis (MS)
g. Juvenile-onset diabetes mellitus
h. Hashimoto's thyroiditis
i. Grave's disease
j. SLE (systemic lupus erythematosus)
k. Sjögren's syndrome
l. Pernicious anemia
m. Chronic active (lupoid) hepatitis
n. Rheumatoid arthritis (RA)
o. Optic neuritis

Moreover, the novel composition or kit according to the invention may be used in the treatment of
1. Pain such as radicular pain, pain associated with radiculopathy, neuropathic pain or sciatica/sciatic pain
2. Organ transplantation (prevention of rejection)
3. Sarcoidosis
4. Necrobiosis lipoidica
5. Granuloma annulare

Moreover, the novel composition or kit according to the invention may be used in the treatment of lupus nephritis, myasthenia gravis, uveitis, refractory uveitis, vernal conjunctivitis, pemphigus vulgaris, or scleroderma.

Psoriasis has been proposed to potentially be associated with Crohn's disease (Najarian D J, Gottlieb A B, Connections between psoriasis and Crohn's disease. J Am Acad Dermatol. 2003 June; 48(6):805-21), celiac disease (Ojetti V et al, High prevalence of celiac disease in psoriasis. Am J Gastroenterol. 2003 November; 98(11):2574-5.), psychiatric or psychological disease, such as depression or a life crisis (Gupta M A, Gupta A K, Psychiatric and psychological co-morbidity in patients with dermatologic disorders: epidemiology and management. Am J Clin Dermatol. 2003; 4(12):833-42. and Mallbris L et al, Psoriasis phenotype at disease onset: clinical characterization of 400 adult cases. J Invest Dermatol. 2005 March; 124(3):499-504.), overweight, diabetes mellitus, excess consumption of alcohol/alcoholism, as well as psoriatic arthritis.

The present invention thus relates in one aspect to a method of treating psoriasis, psoriatic arthritis, neurodermatitis, inflammatory bowel disease, such as Crohn's disease and ulcerative colitis, polyarthritis, multiple sclerosis (MS), juvenile-onset diabetes mellitus, Hashimoto's thyroiditis, Grave's disease, SLE (systemic lupus erythematosus), Sjögren's syndrome, Pernicious anemia, Chronic active (lupoid) hepatitis, Rheumatoid arthritis (RA), lupus nephritis, myasthenia gravis, uveitis, refractory uveitis, vernal conjunctivitis, pemphigus vulgaris, scleroderma, optic neuritis, pain such as radicular pain, pain associated with radiculopathy, neuropathic pain or sciatica/sciatic pain, organ transplantation (prevention of rejection), sarcoidosis, necrobiosis lipoidica or granuloma annulare, which method comprises administering orally to a patient in need thereof, an effective dosage of a controlled release pharmaceutical composition according the invention.

The present invention relates in another aspect to the use of a controlled release pharmaceutical composition according to the invention for the preparation of a medicament for the treatment of psoriasis, psoriatic arthritis, neurodermatitis, inflammatory bowel disease, such as Crohn's disease and ulcerative colitis, polyarthritis, multiple sclerosis (MS), juvenile-onset diabetes mellitus, Hashimoto's thyroiditis, Grave's disease, SLE (systemic lupus erythematosus), Sjögren's syndrome, Pernicious anemia, Chronic active (lupoid) hepatitis, Rheumatoid arthritis (RA), lupus nephritis, myasthenia gravis, uveitis, refractory uveitis, vernal conjunctivitis, pemphigus vulgaris, scleroderma, optic neuritis, pain such as radicular pain, pain associated with radiculopathy, neuropathic pain or sciatica/sciatic pain, organ transplantation (prevention of rejection), sarcoidosis, necrobiosis lipoidica or granuloma annulare.

Furthermore, the invention also relates to treating an individual suffering from one of the conditions in the abovementioned lists, more specifically psoriasis or psoriatic arthritis, with a composition or kit according to the invention, said individual further being in treatment with

a) a topical anti-psoriatic drug such as 1) vitamin D or derivatives thereof (calcipotriol, calcipotriene), 2) a corticosteroid (such as e.g betamethasone, desoximethasone, fluocinolone, momethasone, hydrocortisone aceponate, fluticasone, clobethasol, clobethasone, hydrocortisone butyrate, desonide, triamcinolone or hydrocortisone), 3) tazaroten, 4) ditranol, 5) tacrolimus (FK-506), and other calcineurin inhibitors, such as pimecrolimus or 6) any combination of 1-5 and/or
b) an oral anti-psoriatic drug such as 1) an oral retinoid (such as acitretin or etretinate) combined or not combined with PUVA, 2) cyclosporine and other calcineurin inhibitors, such as ISA247, tacrolimus and pimecrolimus, 3) methotrexate, 4) hydroxyurea, 5) azathioprine, 6) sulphasalazine, 7) a fumarate derivative (such as e.g. Fumaderm® or BG-12), 8) rosiglitazone (Avandia) and other peroxisome proliferator-activated-γ (PPARγ) agonists or modulators, such as pioglitazone, farglitazar, GW1929, GW7845, MC-555, MBX-102/MBX-10, MBX-1828, MBX-2044, CLX-0921, R-483, reglitazar, naveglitazar (LY-519818/LY-818), netoglitazone (MCC-555), CS-7017, troglitazone, ciglitazone, tesaglitazar, isaglitazone, balaglitazone, muraglitazar, TAK-654, LBM642, DRF 4158, EML 4156, T-174, TY-51501, TY-12780, VDO-52 or AMG-131(T131) or any combination of 1-8 and/or
c) a parenterally administered anti-psoriatic drug such as 1) alefacept (Amevive), 2) etanercept (Enbrel), 3) efalizumab (Raptiva), 4) onercept, 5) adalimumab (Humira) or any combination of 1-5 and/or
d) an inhibitor of TNF-a not mentioned in the list under section c) above (e.g. CDP 870 or infliximab (Remicade)), administered via an enteral or parenteral route and/or
e) tisocalicitrate and/or NCX 1022 and/or IDEC-131 and/or MEDI-507, and/or
f) An NSAID or a COX or a LOX inhibitor such as e.g. a COX-2 inhibitor or a COX/5-LOX inhibitor, and/or
g) an anti-diabetic or anti-obesity drug, such as biguanides such as metformin; metformin XR; a sulphonylurea such as chlorpropamide, glipizide, gliclazide, glyburide/glibenclamide or glimepiride; Glucovance (metformin+glyburide); Metaglip (glipizide+metformin); a peroxisome proliferator-activated-γ (PPARγ) agonist or modulator, such as rosiglitazone (Avandia), pioglitazone, farglitazar, GW1929, GW7845, MC-555, MBX-102/MBX-10, MBX-1828, MBX-2044, CLX-0921, R-483, reglitazar, naveglitazar (LY-519818/LY-818), netoglitazone (MCC-555), CS-7017, troglitazone, ciglitazone, tesaglitazar, isaglitazone, balaglitazone, muraglitazar, TAK-654, LBM642, DRF 4158, EML 4156, T-174, TY-51501, TY-12780, VDO-52 or AMG-131(T131); Avandamet (rosiglitazone+metformin); Actos (pioglitazone+metformin); Avandaryl (rosiglitazone maleate+glimepiride); a benzoimidazole such as FK-614; CS-917; TA-1095; ONO-5129; TAK-559; TAK-677/AJ-9667; a d-phenylalanine inducer such as senaglinide; c-3347; NBI-6024; ingliforib; BVT 3498; LY 929; SGLT2 inhibitors; CS 011; BIM 51077; R1438; R1439; R1440; R1498; R1499; AVE 0847; AVE 2268; AVE 5688; AVE 8134; TA-6666; AZD 6370; SSR 162369; TLK-17411; NN 2501; MK 431; KGA-2727; MK-767; CS-872; a beta-3 receptor antagonist such as N-5984; an alpha-glucosidase inhibitor such as acarbose, voglibose or miglitol; a glinitide/meglitinide analogue or carbamoylmethylbensoeic acid derivative such as mitiglinide, repaglinide or nateglinide; a DPP-IV inhibitor such as LAF 237 (vildagliptin), DPP728, P93/01, P32/98, PT-630 or saxagliptin; GLP-1 or GLP-1 analogues, such as exenatide, Exenatide-LAR, liraglutide (NN 2211), ZP 10/AVE 0010, LY 307161, betatropin, CJC-1131, GTP-010, SUN E7001 or AZM 134; pramlinitide acetate; insulin or insulin analogues, such as Humalog (insulin lispro), Humulin, Novolin, Novolog/NovoRapid (insulin aspart), Apidra (insulin glulisine), Lantus (insulin glargine), Exubera, Levemir/NN 304 (insulin detemir), AERx/NN 1998, Insuman, Pulmonary insulin or NN 344; sibutramine or other blockers of the presynaptic reuptake of serotonin and noradrenalin; orlistat and other inhibitors of GI lipases; β3-adrenergic receptor agonists; uncoupling proteins; (specific) antagonists of PPARγ (Peroxisome Proliferator-Activated Receptor γ); insulin secretagogues; rimonabant and other CB1 endocannabinoid receptor antagonists; bupropion; topiramate; leptin agonists; ciliary neurotrophic factor; peptide analogues of the human growth hormone fragment 177-191; cholecystokinin-A receptor agonists; melanocortin-3 agonists; noradrenergic drugs such as phentermine, diethylpropion, phendimetrazine or benzphetamine; or any combination of the anti-diabetic or anti-obesity drugs mentioned above, and/or
h) a drug potentially useful in the treatment of substance abuse e.g. alcohol abuse such as naltrexone, acamprosate, disulphiram or Vivitrex (naltrexone long acting injection), and/or,
i) a drug potentially useful in the treatment of Crohn's disease such as
1. 5-ASA compounds such as sulfasalazine, oral 5-ASA formulations or rectal 5-ASA formulations,
2. glucocorticosteroids such as systemic steroids (e.g. budesonide or prednisolone) or topically acting steroids (e.g. budesonide),
3. antibiotics such as metronidazole or quinolones (e.g. ciprofloxacine, ofloxacine, norfloxacine, levofloxacine or moxifloxacine),
4. immunosuppressives such as azathioprine, 6-mercaptopurine or methotrexate,
5. nutritional therapies such as elemental or polymeric formulas or pre- and probiotics,
6. biological therapies e.g. TNF-α inhibitors such as infliximab, adalimumab, CDP870, CDP571, etanercept or onercept,
7. symptomatic agents such as anti-diarrheals or anti-spasmodics.

Examples of suitable NSAIDs are piroxicam, diclofenac, nabumetone, propionic acids including naproxen, flurbiprofen, fenoprofen, ketoprofen and ibuprofen, fenamates including mefenamic acid, paracetamol, indomethacin, sulindac, meloxicam, apazone, pyrazolones including phenylbutazone, salicylates including aspirin.

Examples of suitable COX-2 inhibitors are rofecoxib (Vioxx), valdecoxib (Bextra), celecoxib (Celebrex), etoricoxib (Arcoxia), lumiracoxib (Prexige), parecoxib (Dynastat), deracoxib (Deram), tiracoxib, meloxicam, nimesolide, (1,1-dimethylheptyl)-6a,7,10,10a-tetrahydro-1-hydroxy-6,6dimethyl-6H-dibenzo[b,d]pyran carboxylic acid (CT-3), 2(5H)-Furanone, 5,5-dimethyl (1-methylethoxy) [4(methylsulfonyl)phenyl]-(DFP); Carprofen (RIMADYL), (Acetyloxy)-benzoic acid, 3-[(nitrooxy)methyl]phenyl ester (NCX4016), P54 (CAS Reg. No. 130996 0) 2,6-Bis(1,1-dimethylethyl) [(E)-(2-ethyl-1,1-dioxo isothiazolidinylidene)methyl]phenoI (S-2474), 5(R)-Thio sulfonamide-3(2H)-benzofuranone (SVT-2016) and N-[3-(Formyl-amino) oxo phenoxy-4H benzopyran yl]methanesulfonamide (“T-614”); or a pharmaceutically acceptable salt thereof.

Examples of suitable COX/5-LOX inhibitors are licofelone (ML-3000 or [2,2-dimethyl-6-(4-chlorophenyl)-7-phenyl-2,3,dihydro-1H-pyrrolizine-5-yl]-acetic acid), di-tert-butylphenols, such as (E)-(5)-(3,5-di-tert-butyl-4-hydroxybenzylidence)-2-ethyl-1,2-isothiazolidine-1,1-dioxide (S-2474), darbufelone or tebufelone and pharmacologically active metabolites as well as derivatives such as dihydro-dimethyl-benzofuran and PGV-20229, dihydro-dimethyl-benzofuran, thiophene derived compounds such as RWJ-63556, N-hydroxy-N-methyl-4-(2,3-bis-(4-methoxyphenyl)-thiophen-5-yl)-butanamide (S19812), methoxytetrahydropyran derivatives, oxygenated xanthones such as 1,3,6,7-Tetrahydroxyxanthone (norathyriol)-pyrazole thiocarbamates, pyrazoles such as modified forms of phenidone containing compounds or the tri-fluoro-benzole substituted pyrazoline derivative BW-755C, tepoxaline and derivatives and di-tert-butylpyrimidines.

It is contemplated that such combination therapy leads to an improved therapeutic response and/or an increased convenience for the individual, compared to said individual being treated without the composition or kit according to the invention.

In a further aspect, the invention relates to a method of reducing side effects associated with oral treatment of any of the conditions a-e and 1-5 listed above, in which method the active pharmaceutical ingredient for treating said condition is used in combination with one or more of the following agents:

a) an antacid such as 1) magnesium hydroxide, 2) magnesium trisilicate, 3) aluminium hydroxyde gel, 3) sodium hydrogencarbonate, 4) magaldrat or any combination of 1-5 and/or
b) a histamine H-2 antagonist such as 1) cimetidine, 2) ranitidine, 3) nizatidine, 4) famotidine, 5) roxatidine, 6) lafutadine or any combination of 1-6 and/or
c) a cytoprotective agent such as 1) sucralfate, 2) tripotassium dictitratobismuthate, 3) carbenoxolone, 4) prostaglandin E-2 analogues such as misoprostol, 5) ecabet, 6) cetraxate HCl, 7) teprenone, 8) troxipide, 9) dicyclomine hydrochloride, 10) sofalcon or any combination of 1-10 and/or
d) a proton pump inhibitor (PPI) such as 1) omeprazole, 2) esomeprazole, 3) lansoproazole, 4) pantoprazole, 5) rabeprazole, 6) CS-526/R-105266, 7) AZD 0865, 8) soraprazan or any combination of 1-8, and/or
e) an NSAID or a COX or a LOX inhibitor such as e.g. a COX-2 inhibitor or a COX/5-LOX inhibitor, and/or
f) pentoxifylline, e.g. at a dose range of from 400 to 800 mg/day.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims. Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are described. All publications mentioned herein are incorporated herein by reference to disclose and describe the methods and/or materials in connection with which the publications are cited. It must be noted that as used herein and in the appended claims, the singular forms “a”, “an”, and “the” include plural referents unless the context clearly dictates otherwise. The patents and publications discussed herein are provided solely for their disclosure prior to the filing date of the present application. Nothing herein is to be construed as an admission that the present invention is not entitled to antedate such patent or publication by virtue of prior invention. Further, the dates of publication provided may be different from the actual publication dates which may need to be independently confirmed. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. The figures shown herein are not necessarily drawn to scale, with some components and features being exaggerated for clarity.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

### EXAMPLES

### Example 1

In a granulation process 50 g DMF (dimethyl fumarate in the following DMF) is mixed with 12 g Ethylcellulose (e.g. Ethocel® NF premium) and 3 g Polyethylenglycole 400 which is dissolved in 150 ml Ethanol 96%, passed through a 1.0 mm sieve, dried at 50° to 60° C. over 30 min and again passed through a sieve 1.0 mm. A placebo granulate is prepared as follows: Tablettose® and Avicel® 102 are mixed in equal shares and granulated with 2% povidone (e.g. Kollidon® 25) dissolved in water (q.s.), passed through a 1.0 mm sieve, dried at 50° to 60° C. over 30 min and again passed through a 1.0 mm sieve. 60 parts of the DMF-granulate and 38 parts of the placebo-granulate are mixed for 30 minutes in a Turbula Shaker Mixer. One part Aerosil® 200 and one part magnesium stearate are added and the blend is mixed again for 5 minutes.

The blend is compressed to tablets with a diameter of 10 mm, a weight of about 260 mg and a hardness of about 50 N. The tablets are enteric coated using the processes described in Example 6.

### Example 2

In a granulation process 50 g DMF is mixed with 12 g Ethylcellulose (e.g. Ethocel® NF premium) and 3 g Polyethylenglycole 400 which is dissolved in 150 ml Ethanol 96%, passed through a 1.0 mm sieve, dried at 500 to 60° C. over 30 min and again passed through a sieve 1.0 mm. A placebo granulate is prepared as follows: Tablettose® and Avicel® 102 are mixed in equal shares and granulated with 2% povidone (e.g. Kollidon® 25) dissolved in water (q.s.), passed through a 1.0 mm sieve, dried at 50° to 60° C. over 30 min and again passed through a 1.0 mm sieve. 60 parts of the DMF-granulate and 37 parts of the placebo-granulate are mixed for 30 minutes in a Turbula Shaker Mixer. One part carboxymethylcellulose (e.g. Ac-Di-Sol®), one part Aerosil® 200 and one part magnesium stearate are added and the blend is mixed again for 5 minutes.

The blend is compressed to tablets with a diameter of 10 mm, a weight of about 260 mg and a hardness of about 50 N. The tablets are enteric coated using the processes described in Example 6.

### Example 3

In a granulation process 50 g DMF is mixed with 50 g Eudragit RS D30, 0.75 g Dibutylsebacate and 0.0075 g Tween 80, passed through a 1.0 mm sieve, dried at 500 to 60° C. over 60 min and again passed through a sieve 1.0 mm. A placebo granulate is prepared as follows: Tablettose® and Avicel® 102 are mixed in equal shares and granulated with 20% povidone (e.g. Kollidon® 25) dissolved in water (q.s.), passed through a 1.0 mm sieve, dried at 50° to 60° C. over 30 min and again passed through a 1.0 mm sieve. 60 parts of the DMF-granulate, 37 parts of the placebo-granulate and one part carboxymethylcellulose (e.g. Ac-Di-Sol®) Acdisol are mixed for 30 minutes in a Turbula Shaker Mixer. One part Aerosil® 200 and one part magnesium stearate are added and the blend is mixed again for 5 minutes.

The blend is compressed to tablets with a diameter of 10 mm, a weight of about 260 mg and a hardness of about 50 N. The tablets are enteric coated using the processes described in Example 6.

### Example 4

In a granulation process 50 g DMF is mixed with 50 g Eudragit RS D30, passed through a 1.0 mm sieve, dried at 50° to 60° C. over 60 min and again passed through a sieve 1.0 mm. A placebo granulate is prepared as follows: Tablettose® and Avicel® 102 are mixed in equal shares and granulated with 2% povidone (e.g. Kollidon® 25) dissolved in water (q.s.), passed through a 1.0 mm sieve, dried at 50° to 60° C. over 30 min and again passed through a 1.0 mm sieve. 60 parts of the DMF-granulate, 37 parts of the placebo-granulate and one part carboxymethyl cellulose (e.g. Ac-Di-Sol®) are mixed for 30 minutes in a Turbula Shaker Mixer. One part Aerosil® 200 and one part magnesium stearate are added and the blend is mixed again for 5 minutes.

The blend is compressed to tablets with a diameter of 10 mm, a weight of about 260 mg and a hardness of about 50 N. The tablets are enteric coated using the processes described in Example 6.

### Example 5

In a granulation process 50 g DMF is mixed with 50 g Eudragit RS D30, passed through a 1.0 mm sieve, dried at 50° to 60° C. over 60 min and again passed through a sieve 1.0 mm. A placebo granulate is prepared as follows: Tablettose® and Avicel® 102 are mixed in a ratio of 1:2 and granulated with 2% povidone (e.g. Kollidon® 25) dissolved in water (q.s.), passed through a 1.0 mm sieve, dried at 50° to 60° C. over 30 min and again passed through a 1.0 mm sieve. 60 parts of the DMF-granulate, 37 parts of the placebo-granulate and one part carboxymethylcellulose (e.g. Ac-Di-Sol® are mixed for 30 minutes in a Turbula Shaker Mixer. One part Aerosil® 200 and one part magnesium stearate are added and the blend is mixed again for 5 minutes.

The blend is compressed to tablets with a diameter of 10 mm, a weight of about 260 mg and a hardness of about 50 N. The tablets are enteric coated using the processes described in Example 6.

### Example 6

### Enteric Coating of Tablets

The tablets may be cured before the enteric coating by coating with a solution of 5% Kollidon 25 in an amount of 1 to 2 mg/cm² tablet surface. The PVP-Solution is, depending on the process (fluid bed, drageecauldron), water-, water/isopropanol- or isopropanol-based.

The tablets are enteric coated by spraying with a Eudragit L 30D dispersion in an amount of 5 mg/cm². In the following formulation parts of the titan dioxide can be replaced with a coloured pigment.

Suspension for enteric coating:

| | |
|---|---|
| Eudragit L 30D-55 | 100.0 g |
| Triethyl citrate | 3.0 g |
| Talkum | 7.5 g |
| Titan dioxide | 7.5 g |
| Water | 82.0 g |

1. A pharmaceutical composition comprising as an active substance 40% to 50% by weight of one or more fumaric acid esters selected from di-(C₁-C₅)alkylesters of fumaric acid and mono-(C₁-C₅)alkylesters of fumaric acid, or a pharmaceutically acceptable salt thereof, 8% to 15% by weight of pharmaceutically acceptable polymer(s) and optionally pharmaceutically acceptable excipients or additives.

2. The composition according to claim 1, wherein the amount of fumaric acid ester(s) is 42% to 48% by weight.

3. The composition according to claim 1, wherein the amount of pharmaceutically acceptable polymer(s) is 9% to 14% by weight.

4. The composition according to claim 1 further comprising from 0.1% to 5% by weight hydrophilic excipient(s).

5. The composition according to claim 1, wherein the pharmaceutically acceptable polymer is one or more selected from the group consisting of ethylcellulose, methacrylic acid copolymer, and acrylic acid copolymer.

6. The composition according to claim 1, wherein the pharmaceutically acceptable polymer is one or more selected from the group consisting of ammonio methacrylate copolymer type A, ammonio methacrylate copolymer B, methacrylic acid copolymer A, methacrylic acid copolymer B, polyvinyl acetate polymer and methacryl acetate polymer.

7. The composition according to claim 5, wherein the pharmaceutically acceptable polymer is ethyl cellulose.

8. The composition according to claim 6, wherein the pharmaceutically acceptable polymer is ammonio methacrylate copolymer B.

9. The composition according to claim 4, wherein the hydrophilic excipient is polyethylene glycol.

10. The composition according to claim 4, wherein the hydrophilic excipient is hydroxyl propyl cellulose.

11. The composition according to claim 1, wherein the fumaric acid ester is formulated for release in a controlled dose over a predetermined time period,
wherein about 50% w/w of the total amount of the fumaric acid ester contained in the composition is released within about 3 hours after oral administration.

12. The composition according to claim 1, wherein the fumaric acid ester is formulated for release in a controlled dose over a predetermined time period,
wherein about 70% w/w of the total amount of the fumaric acid ester is released within about 4 hours after oral administration.

13. The composition according to claim 1, wherein the fumaric acid ester is formulated for release in a controlled dose over a predetermined time period,
wherein about 85% w/w of the total amount of the fumaric acid ester is released within about 5 hours after oral administration.

14. The composition according to claim 1, wherein the fumaric acid ester is formulated for release in a controlled dose over a predetermined time period,
wherein about 95% w/w of the total amount of the fumaric acid ester is released within about 6 hours after oral administration.

15. The composition according to claim 1, wherein the release has zero-order, first-order or square-root (Higuchi's equation) kinetics release profile.

16. The composition according to claim 15, wherein the release has a square-root (Higuchi's equation) kinetics release profile.

17. The composition according to claim 1, which—upon oral administration and in comparison to that obtained after oral administration of Fumaderm® tablets in an equivalent dosage—gives a reduction in GI related side effects.

18. The composition according to claim 1 in the form of a tablet.

19. The composition according to claim 1 having an enteric coating.

20. The composition according to claim 1, wherein the fumaric acid ester is selected from the group consisting of dimethylfumarate, diethylfumarate, dipropylfumarate, dibutylfumarate, dipentylfumarate, methyl-ethyl-fumarate, methyl-propylfumarat, methyl-butylfumarat, methyl-pentylfumarate, monomethylfumarate, monoethylfumarate, monopropylfumarate, monobutylfumarate, and monopentylfumarate, including pharmaceutically acceptable salts thereof.

21. The composition according to claim 1, wherein the fumaric acid ester is a mono-(C₁-C₅)alkylester of fumaric acid that is present in the form of a pharmaceutically acceptable salt.

22. The composition according to claim 21, wherein the salt is a metal salt such as a salt selected from the group consisting of alkali metal salts and alkaline earth metal salts.

23. The composition according to claim 22, wherein the salt is selected from the group consisting of sodium, potassium, calcium, magnesium and zinc salt.

24. The pharmaceutical composition according to of claim 1, comprising dimethylfumarate as the active substance.

25. The pharmaceutical composition according to claim 1 comprising monomethylfumarate as the active substance.

26. The composition according to claim 25, wherein monomethylfumarate is present in the form of a salt selected form the group consisting of sodium, potassium, calcium, magnesium and zinc salt.

27. The composition according to claim 1 for administration once, twice or three times daily.

28. The composition according to claim 27 for administration once daily.

29. The composition according to claim 27 for administration twice daily.

30. The composition according to claim 1, wherein the amount of one or more fumaric acid esters selected from di-(C₁-C₅)alkylesters of fumaric acid and mono-(C₁-C₅)alkylesters of fumaric acid or a pharmaceutically acceptable salt thereof, in a dosage form is from 90 mg to 500 mg active substance.

31. A method of treating psoriasis, psoriatic arthritis, neurodermatitis, inflammatory bowel disease, such as Crohn's disease and ulcerative colitis, polyarthritis, multiple sclerosis (MS), juvenile-onset diabetes mellitus, Hashimoto's thyroiditis, Grave's disease, SLE (systemic lupus erythematosus), Sjögren's syndrome, Pernicious anemia, Chronic active (lupoid) hepatitis, Rheumatoid arthritis (RA), lupus nephritis, myasthenia gravis, uveitis, refractory uveitis, vernal conjunctivitis, pemphigus vulgaris, scleroderma, optic neuritis, pain such as radicular pain, pain associated with radiculopathy, neuropathic pain or sciatica/sciatic pain, organ transplantation (prevention of rejection), sarcoidosis, necrobiosis lipoidica or granuloma annulare, which method comprises administering orally to a patient in need thereof, an effective dosage of a pharmaceutical composition according to claim 1.

32. The pharmaceutical composition according to claim 1 for the preparation of a medicament in the treatment of psoriasis, psoriatic arthritis, neurodermatitis, inflammatory bowel disease, such as Crohn's disease and ulcerative colitis, polyarthritis, multiple sclerosis (MS), juvenile-onset diabetes mellitus, Hashimoto's thyroiditis, Grave's disease, SLE (systemic lupus erythematosus), Sjögren's syndrome, Pernicious anemia, Chronic active (lupoid) hepatitis, Rheumatoid arthritis (RA), lupus nephritis, myasthenia gravis, uveitis, refractory uveitis, vernal conjunctivitis, pemphigus vulgaris, scleroderma, optic neuritis, pain such as radicular pain, pain associated with radiculopathy, neuropathic pain or sciatica/sciatic pain, organ transplantation (prevention of rejection), sarcoidosis, necrobiosis lipoidica or granuloma annulare.
